(19) United States
(12) Patent Application Publication
(10) Pub. No.: US 2024/0268633 A1
(43) Pub. Date: Aug. 15, 2024

(54) MEDICAL SYSTEMS AND DEVICES WITH DISPOSABLE FEATURES

(71) Applicant: Boston Scientific Scimed, Inc., Maple Grove, MN (US)

(72) Inventors: Louis J. BARBATO, Franklin, MA (US); Kirsten VIERING, Newton, MA (US); Eric F. BROWN, Wilmington, MA (US)

(73) Assignee: Boston Scientific Scimed, Inc., Maple Grove, MN (US)

(21) Appl. No.: 18/437,673

(22) Filed: Feb. 9, 2024

Related U.S. Application Data
(60) Provisional application No. 63/484,241, filed on Feb. 10, 2023.

(51) Int. Cl. A61B 1/00

(52) U.S. Cl. CPC A61B 1/00105; A61B 1/00066; A61B 1/0008; A61B 1/00128

(57) ABSTRACT

A medical device, comprising a handle including a port, a shaft extending distally from the handle, wherein the shaft includes a channel extending along at least a portion of a length of the shaft, wherein the channel includes a longitudinal opening on a radially outer surface of the shaft, and a removable portion extending through the channel, wherein the removable portion includes a first lumen, and wherein the removable portion is configured to be separated from the handle and the shaft.

### CROSS-REFERENCE TO RELATED APPLICATIONS

This application claims the benefit of priority to U.S. Provisional Application No. 63/484,241, filed on Feb. 10, 2023, which is incorporated by reference herein in its entirety.

### TECHNICAL FIELD

This disclosure relates generally to medical systems and devices. More particularly, the disclosure relates to medical systems and devices including removable, disposable features.

### BACKGROUND

Medical imaging devices, such as endoscopes, allow physicians to visualize tissues, anatomy, and pathology in a minimally invasive way. These imaging devices are typically cleaned and reused multiple times. Sterilization of such imaging devices is imperative to avoid cross-contamination between patients. Unfortunately, insufficient sterilization of imaging devices is a persistent, and hazardous problem. Therefore, a need exists for medical imaging devices having single-use elements.

### SUMMARY OF THE DISCLOSURE

According to an embodiment, a medical device may comprise a handle including a port, a shaft extending distally from the handle, wherein the shaft includes a channel extending along at least a portion of a length of the shaft, wherein the channel includes a longitudinal opening on a radially outer surface of the shaft; and a removable portion extending through the channel, wherein the removable portion includes a first lumen, and wherein the removable portion is configured to be separated from the handle and the shaft.

In an embodiment, the channel of the shaft may be off center from a central longitudinal axis of the shaft. The channel may be configured to prevent the removable portion from passing through the longitudinal opening of the channel.

In an embodiment, the longitudinal opening of channel may include a notch, a groove, or a slit extending along an entire length of the shaft.

In an embodiment, the shaft may include a distal end, wherein the distal end includes an abutment outlining a distal opening of the channel, and wherein the abutment is configured to inhibit distal translation of the removable portion past the distal end.

In an embodiment, the removable portion may include a second lumen.

In an embodiment, the removable portion may consist of the first lumen and the second lumen.

In an embodiment, the removable portion may include a proximal adapter, wherein the proximal adapter includes a first port in fluid communication with the first lumen of the removable portion. The proximal adapter may be configured to releasably engage the port of the handle. The proximal adapter may include an actuator configured to release the proximal adapter from the port of the handle. The proximal adapter may be a y-shaped adapter including the first port of the proximal adapter and a second port of the proximal adapter.

In an embodiment, the first lumen may include an elevator configured to adjust an orientation of a tool or instrument inserted within the first lumen.

In an embodiment, the removable portion may comprise a substantially teardrop-shaped cross-section.

In an embodiment, the removable portion may include a second lumen, and only the removable portion may be configured to receive fluid via the first lumen and/or the second lumen.

In an embodiment, the removable portion may be configured to be completely removed from the handle and the shaft by pulling the removable portion proximally from the port of the handle.

According to another embodiment, a medical device may comprise a handle including a cavity, a shaft extending distally from the handle, wherein the shaft includes a channel extending along at least a portion of a length of the shaft, wherein the channel includes a longitudinal opening on a radially outer surface of the shaft, and a removable portion extending through the cavity and the channel, wherein the removable portion includes a first lumen, and wherein the removable portion is configured to be separated from the handle and the shaft.

In an embodiment, the cavity may be partially open thereby exposing at least a portion of the removable portion.

In an embodiment, the removable portion may include a body and an elongated portion, and the body may include a port configured to receive a medical instrument.

According to another embodiment, a medical device may comprise a handle including a port, a shaft extending distally from the handle, wherein the shaft includes a channel extending along at least a portion of a length of the shaft, and a removable portion extending throughout the channel, wherein the removable portion includes at least one lumen, and wherein the removable portion is configured to be removed from the handle and the shaft via the port, and a liner positioned between the channel and the removable portion, wherein the liner is configured to transition between a deflated state and an inflated state.

In an embodiment, the liner may be fixed to at least a portion of a surface of the removable portion and the removable portion and the liner may be configured to be completely removed from the handle and the shaft by pulling the removable portion proximally from the port of the handle.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is a partial cross-sectional view of a system including a medical device, according to some aspects of the disclosure.

FIGS. 2A and 2B are perspective views of an aspect of the medical device of FIG. 1.

FIGS. 2C and 2D are perspective views of another aspect of the medical device of FIG. 1.

FIGS. 3A and 3B are perspective views of a medical device, according to another aspect of the disclosure.

FIG. 4 is a partial cross-sectional view of a system including a medical device, according to another aspect of the disclosure.

FIGS. 5A and 5B are perspective views of an aspect of the medical device of FIGS. 3A and 3B.

FIGS. 6A-6C are perspective views of a medical device, according to another aspect of the disclosure.

### DETAILED DESCRIPTION

Reference will now be made in detail to aspects of the disclosure, examples of which are illustrated in the accompanying drawings. Wherever possible, the same or similar reference numbers will be used through the drawings to refer to the same or like parts. The term “distal” refers to a location or portion of a medical device farthest away from a user of the device, e.g., when introducing a device into a subject (e.g., patient). By contrast, the term “proximal” refers to a location or portion closest to the user, e.g., when placing the device into the subject.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of 10% in a stated value or characteristic.

Embodiments of this disclosure include medical systems and devices including a removable portion. In some examples, the removable portion may be disposed of after single use (i.e., may be disposable). Said removable portion may include elements of an imaging device that may be difficult to clean/sterilize and may be common sources of contamination. Such elements may include one or more of a working channel, elevator features (e.g., those that may be present in the working channel), fluidic/suction channels, and/or other elements which may come in contact with fluids (e.g., bodily fluids), anatomy, etc., which may contaminate the elements. This removable portion may be a separate component from a handle and a shaft of an exemplary medical device so that the handle and the shaft may be sealed and kept separate from the one or more channels/features of said removable portion. Removable portions may also be electronically tagged or marked via any suitable manner to prevent re-use. The handle, the shaft, and features of the shaft (e.g., camera(s), light(s), etc.) may be cleaned and sterilized, and may be re-used with a new removable portion inserted into the exemplary medical device. Such an exemplary medical device may help reduce the cost of endoscopic procedures, reduce the hazardous materials waste, and reduce the risk of cross-contamination between patients. In other examples, a removable portion of another exemplary medical device may include a camera, lights, etc. In such examples, a disposable shaft may include one or more channels (e.g., working channel, air/water channel, and/or suction channel), elevator features, etc. The removable portion of such an exemplary device may be cleaned/sterilized and re-used, while the remaining aspects of such a medical device (e.g., the handle, the shaft, etc.) may be disposed of after a single use. Such exemplary medical systems and devices are discussed in further detail below.

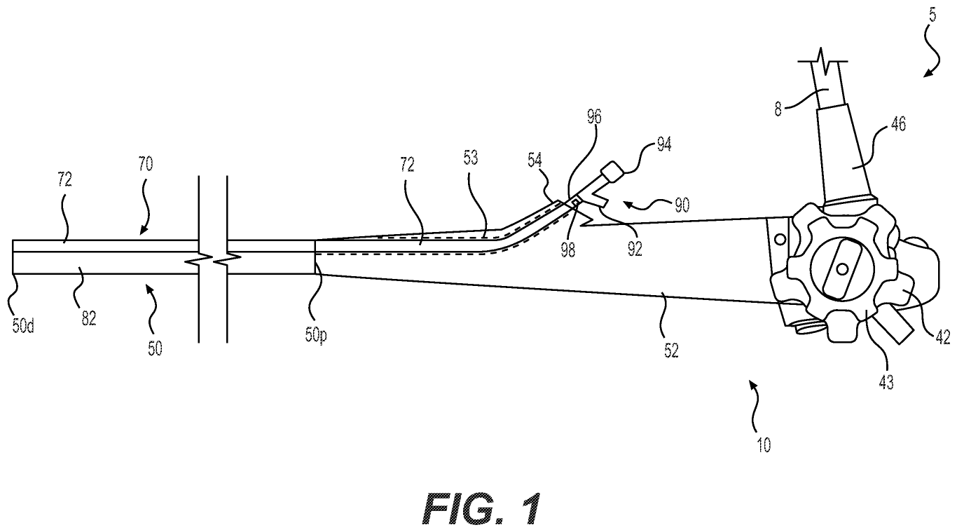

Referring to FIG. 1, a medical system 5 including a medical device 10, e.g., an endoscope, according to one or more embodiments of the disclosure is shown. Medical device 10 includes a flexible shaft 50 (e.g., a catheter) and a handle 52 connected at a proximal end of flexible shaft 50.

Handle 52 is not particularly limited, and may be configured for actuating or otherwise controlling features of medical system 5 and/or one or more tools or devices associated with medical system 5. The handle 52 as shown includes first and second actuators 42, 43, which control articulation of shaft 50, e.g., an articulation joint at or proximate a distal end of shaft 50. For example, the actuators 42, 43 may control movement of the shaft 50 in multiple directions, e.g., movement along different planes. Actuators 42, 43, may include, for example, rotatable knobs that rotate to push/pull cables or wires coupled to the shaft 50. For example, one or more cables or wires may comprise medical grade plastic or metal, and may extend distally from a respective actuator 42, 43 to connect to flexible shaft 50 to control movement thereof. Distal ends of the cables or wires may extend through shaft 50 and terminate at an articulation joint and/or a distal tip of shaft 50. For example, one or more cables or wires may be connected to an articulation joint, and rotation of actuators 42, 43 may control the cables or wires to move the articulation joint and/or the distal end of shaft 50, e.g., along multiple directions. According to some aspects of the disclosure, one or more electrical cables (not shown) may extend from the proximal end of system 5 to the distal end of shaft 50 and may provide electrical controls to imaging, lighting, and/or other electronics at the distal end of shaft 50. Electrical cables may carry imaging signals received at the distal end of shaft 50 to be processed and/or displayed on a display. Handle 52 may also include at least one port, such as port 54 shown in the example of FIG. 1, for introducing and/or removing tools, fluids, pressure (e.g., pulling negative pressure or suction) or other materials from the subject (e.g., patient) via a removable portion 70 (discussed in further detail below). Handle 52 may include a lumen 53 which may extend between an opening of port 54 and a proximal opening of a channel 83 of shaft 50 (discussed in further detail below). Thus, lumen 53 may define a passage of any suitable dimension through which removable portion 70 may be guided towards channel 83.

Handle 52 also includes port 46 which may be detachably connected to an umbilicus 8 for introducing wiring for electronic components positioned at a distal end of shaft 50. Thus, in some embodiments, umbilicus 8 may be detached from port 46 and handle 52 when handle 52 is to be disposed of. Handle 52 may further include additional lumen (not shown) extending between port 46 and shaft 50, providing a passage for any wiring associated with the electronic components of shaft 50. In alternatives, handle 52 may be fixedly attached to umbilicus 8 and port 46 may be omitted.

As shown in FIG. 1, shaft 50 includes a reusable portion 82 and a removable portion 70. Reusable portion 82 may extend between a distal end 50d, and a proximal end 50p. Proximal end 50p may be coupled to a distal portion of handle 52 via any suitable means, and may include an opening (not shown) which may be in communication with a lumen or the cavity of handle 52.

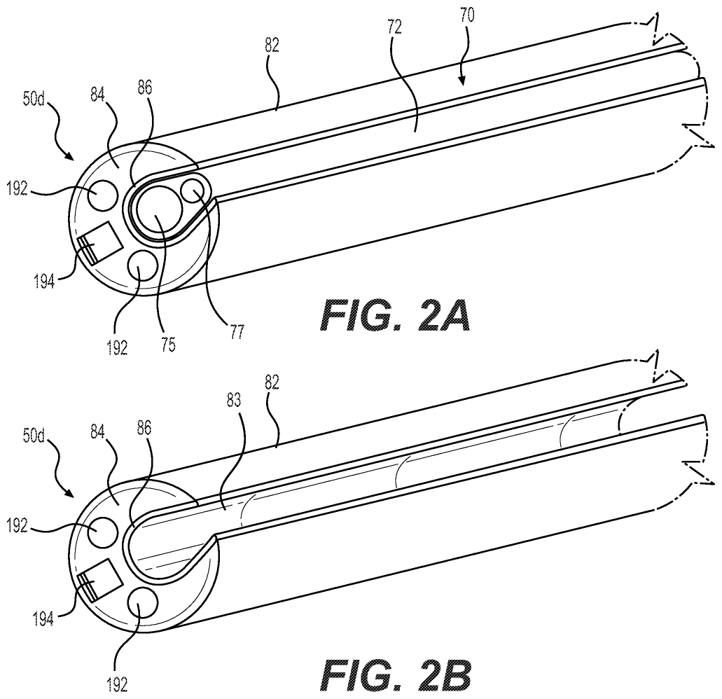

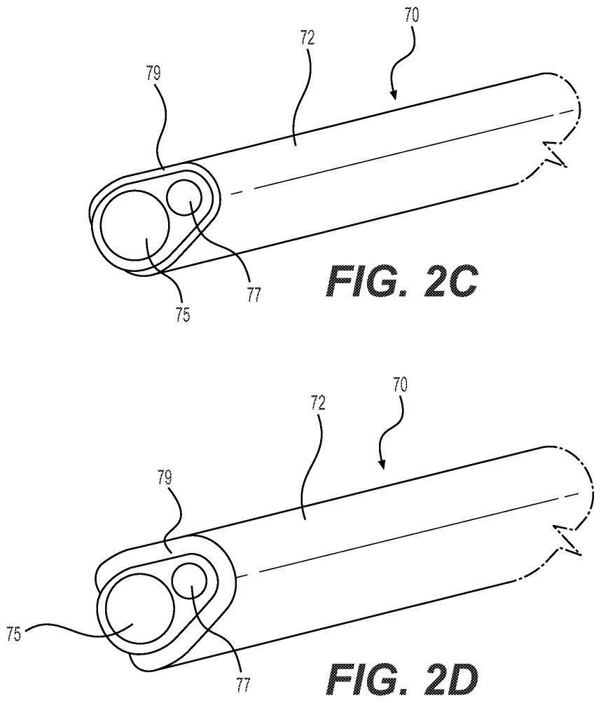

Reusable portion 82 is not particularly limited and may comprise any suitable biocompatible material. As shown in FIGS. 2A and 2B, reusable portion 82 may include a distal face 84 including various electronic features including an imager (e.g., a camera) 194, and/or light features 192 (e.g., LED). The various cables/wirings associated with each of imager 194 and light features 192 may extend throughout a length of shaft portion 82, through the opening of proximal end 50p, through a lumen or cavity of handle 52, and towards port 46 and umbilicus 8. In some examples, reusable portion 82, as shown in FIGS. 2A and 2B, may be without any channels or lumens configured to receive medical tools/instruments, suction, or fluidics. Thus, reusable portion 82 (along with handle 52) may be easily cleaned, sterilized, and re-used. Reusable portion 82 may further include a longitudinal channel 83 (shown in FIG. 2B), which may extend between distal end 50d and proximal end 50p. Channel 83 is not particularly limited and may be of any suitable dimension configured to receive an elongated portion 72 of removable portion 70. In some examples, reusable portion 82 may include a longitudinal opening extending between distal end 50d and proximal end 50p and partially exposing the passage defined by channel 83. For example, channel 83 may be open on a radially outer side of channel 83, as well as at a distal end of channel 83. In such examples, channel 83 may include a notch, groove, or slit formed in an outer surface (e.g., radially outer surface) of reusable portion 82, longitudinally along reusable portion 82. Thus, channel 83 may be “partially open”, exposing a portion of a surface of elongated portion 72 of removable portion 70 as shown in FIG. 2A (or channel 83 when removable portion 70 is removed as shown in FIG. 2B). However, despite being partially open, channel 83 may be configured to retain removable portion 70 by spanning around a sufficient degree/amount of a perimeter of elongated portion 72, thereby retaining elongated portion 72 in place (preventing elongated portion 72 from passing through the radial opening in reusable portion 82). As a result, the partially open channel may provide a user an option of inserting elongated portion 72 of disposable portion 70 through port 54 and channel 53 until disposable portion 70 reaches distal end 50d, or an option of inserting elongated portion 72 of disposable portion 70 at distal end 50d and pressing elongated portion 70 into channel 83 and through proximal end 50d and channel 53. Channel 83 may be rigid enough to hold disposable portion 70 in place, but flexible enough to allow disposable portion 70 to be pressed into channel 83 with enough force. Also, the partially open channel 83 may provide design flexibility in disposable portion 70 such that the size and shape of elongated portion 72 is not constrained by a closed channel. In other examples, channel 83 may be closed thereby encapsulating a whole perimeter (e.g., a whole circumference where elongated portion 72 has a circular cross-section) of elongated portion 72.

The cross-sectional shape/size of channel 83 is not particularly limited and may correspond to the cross-sectional shape/size of elongated portion 72. In other words, the cross-sectional shape of channel 83 may be keyed to/complementary/mating to the cross-sectional shape of elongated portion 72. In some examples, channel 83 may have a C-shaped, U-shaped, teardrop-shaped, or circularly-shaped cross-section, which may be suitable for retaining or clamping elongated portion 172, but is not necessarily limited thereto.

Distal face 84 of reusable portion 82 may further include an abutment 86 outlining a distal opening of channel 83, and abutment 86 may be configured to inhibit further distal translation of removable portion 70 past distal face 84. For example, abutment 86 may include a projection that extends inward along a perimeter of channel 83, effectively causing a distal opening of channel 83 to have a smaller perimeter than (a) more proximal portions of channel 83 and (b) elongated portion 72.

Removable portion 70 includes elongated portion 72 that may extend throughout a length of channel 83. A material of elongated portion 72 is not particularly limited and may comprise any suitable biocompatible material. As shown in FIGS. 2C and 2D, elongated portion 72 may include a working channel 75 and a second channel 77. Working channel 75 is not particularly limited and may be any suitable channel configured to receive at least one medical instrument, tool, or accessory. Likewise, second channel 77 is not particularly limited and may be any suitable channel configured to receive and/or deliver suction, fluid, etc. Elongated portion 72 is not necessarily limited to channels 75 and 77, and, in some examples, may include additional lumens or may be limited to just one lumen. Relative positions and sizes of channels 75, 77 shown in FIGS. 2C and 2D are merely exemplary, and any suitable arrangement may be utilized.

Moreover, elongated portion 72 may include an elevator feature (not shown) for changing an orientation of a medical instrument inserted in working channel 75 and/or another lumen configured to receive a medical instrument. Such an elevator feature may also be referred to as a swing stand, pivot stand, raising base, or any other suitable term. Said elevator may be pivotable via an actuation wire or another control element (not shown) that extends throughout a length of elongated portion 72 (e.g., via a separate lumen of elongated portion 72) and to a respective actuator or control (e.g., a lever) (not shown) positioned on handle 52 or removable portion 70.

In some examples, removable portion 70 may be devoid of any electronics or circuitry, and removable portion 70 may be disposable after procedure. The cross-section of elongated portion 72 may be circularly-shaped, oval-shaped, teardrop-shaped, ovoid-shaped, etc., but is not necessarily limited thereto. As discussed above, the cross-sectional shape/size of elongated portion 72 may correspond to the cross-sectional shape/size of channel 83 so that elongated portion 72 may securely fit within channel 83. In other words, elongated portion 72 may mate with/be keyed to channel 83.

As shown in FIGS. 2C and 2D, elongated portion 72 may further include a liner 79 surrounding at least a portion of an outer surface of elongated portion 72. The amount of said surface covered by liner 79 is not particularly limited, as liner 79 may span around a portion or an entirety of a perimeter of elongated portion 72 and may extend across a portion or an entirety of a length of elongated portion 72. Liner 79 may define one or more inflatable chambers and each of the one or more inflatable chambers may longitudinally extend along a length of elongated portion 72. A proximal portion of liner 79 may include one or more openings or valves (not shown) configured to receive and/or expel air or fluid from an external source. Thus, elongated portion 72, with liner 79, may transition between a default, deflated state, as shown in FIG. 2C, and an inflated state, as shown in FIG. 2D. Elongated portion 72 may be inserted into channel 83 of reusable portion 82 while in the deflated state, thereby allowing for easier insertion of elongated portion 72 through port 54 and channel 83. Elongated portion 72 may be inflated while within channel 83, thereby increasing the pressure between a surface of channel 83 and liner 79 and further securing elongated portion 72 within channel 83. Following a procedure, elongated portion 72 may revert to the deflated state while within channel 83, so that the pressure between the surface of channel 83 and liner 79 is decreased, thereby allowing elongated portion 72 to be more easily removed from channel 83 and through port 54. In other examples, an inflatable liner may surround a surface of channel 83 in addition to, or instead of, liner 79 surrounding elongated portion 72. Such an inflatable liner may function similarly to that of liner 79.

As shown in FIG. 1, removable portion 70 may additionally include a proximal adapter 90. Proximal adapter 90 is not particularly limited and may be of any suitable biocompatible material, and in some embodiments, may be of a softer material than that of handle 52. Proximal adapter 90 is coupled to a proximal end of elongated portion 72 via any suitable manner and may be any adapter, e.g., a Y-port adapter (an adapter/port having a Y-shape), configured to releasably engage port 54 of handle 52. Proximal adapter 90 may include a first port 92 in fluid communication with second channel 77, a second port 94 in communication with working channel 75, and a connective end 96 configured to releasably couple to port 54. Port 92 is not particularly limited, and may be configured to receive any tubing in connection with a suction or fluid source, and may include additional controls or buttons (not shown) configured to control delivery of suction or fluid. Likewise, port 94 is not particularly limited, and may be configured to receive additional medical tools, instruments, and/or accessories.

Connective end 96 is also not particularly limited and may include any suitable features for coupling to port 54. For example, connective end 96 may include at least one mating feature (not shown) (e.g., a peg of any suitable shape (rectangle, square, etc.), and port 54 may include a corresponding mating feature (not shown) (e.g., a detent, opening, receiving feature, etc.) configured to receive the at least one mating feature of end 96. Alternatively, connective end 96 may include at least one mating feature configured to receive the at least one mating feature of port 54. Moreover, connective end 96 or port 54 may include a biasing mechanism (e.g., a spring locking detent) (not shown), which may be configured to bias the coupling between connective end 96 and port 54 in a locked and secured state. In some examples, proximal adapter may further include an actuator 98 (e.g., a button, a control, etc.) configured to lock and/or release the corresponding mating features of connective end 96 and port 54.

Elongated portion 72 may fit within and extend throughout channel 83 via an opening of port 54, and removable portion 70 may be secured (and locked) to handle 52 via the coupling between connective end 96 and port 54. Removable portion 70 may also be completely removed from reusable portion 82 and handle 52 by releasing connective end 96 from port 54 (via actuator 98, in some examples) and pulling elongated portion 72 out of channel 83 and through port 54. Thus, a user may remove and dispose removable portion 70 after use in a procedure, and may introduce a new removable portion 70 into reusable portion 82 and handle 52 (after cleaning and sterilization of reusable portion 82 and handle 52) for a subsequent procedure.

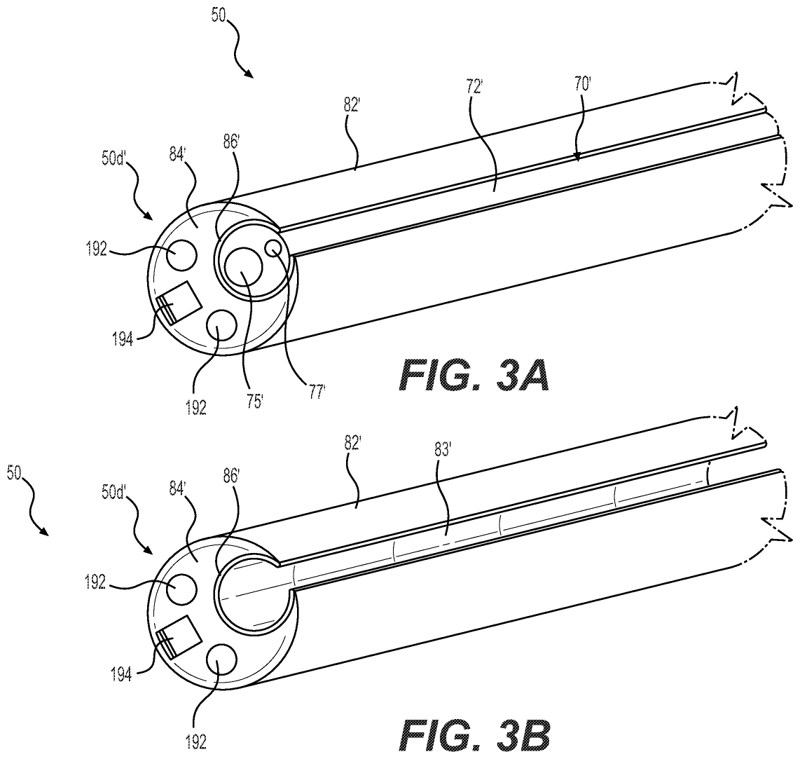

FIGS. 3A and 3B show another exemplary shaft 50′. Shaft 50′ is similar to shaft 50 in many respects, and like reference numerals (e.g., reusable portion 82′ and reusable portion 82) refer to like parts. Like shaft 50, shaft 50′ includes a reusable portion 82′ and a removable portion 70′. Reusable portion 82′ includes a distal face 84′ including various electronic features including an imager (e.g., a camera) 194, and/or light features 192 (e.g., one or more LEDs), and, in some embodiments, may be without any channels or lumens configured to receive medical tools/instruments, suction, or fluidics. Reusable portion 82′ further includes a channel 83′ (as shown in FIG. 3B) extending between a distal end 50d′ of shaft 50′ and a proximal end of shaft 50′ (not shown). Unlike channel 83 which has a U or C-shaped cross-section, channel 83′ may have a round, circular cross-section that is configured to receive an elongated portion 72′ of removable portion 70′. As shown in FIG. 3B, channel 83 may be off center from a central, longitudinal axis of shaft 50′, as channel 83 may extend radially inwards from a surface of shaft 50′.

Removable portion 70′ includes elongated portion 72′ that may extend throughout a length of channel 83′. Like elongated portion 72, elongated portion 72′ may include a working channel 75′ and a second channel 77′, as well as other additional features (e.g., an elevator feature), but may be devoid of any electronics or circuitry. Unlike elongated portion 72, the cross-section of elongated portion 72′ may be round/circular so it may fit within and extend through channel 83′, which, as noted above, has a corresponding circular cross-section. The dimensions (e.g., a diameter, an area of a cross-section, etc.) of elongated portion 72′ are not particularly limited and elongated portion 72′ may have any suitable dimensions that correspond to those of channel 83′. In some embodiments, an area of a cross section of elongated portion 72′ may be less than an area of a cross section of elongated portion 72, or vice versa.

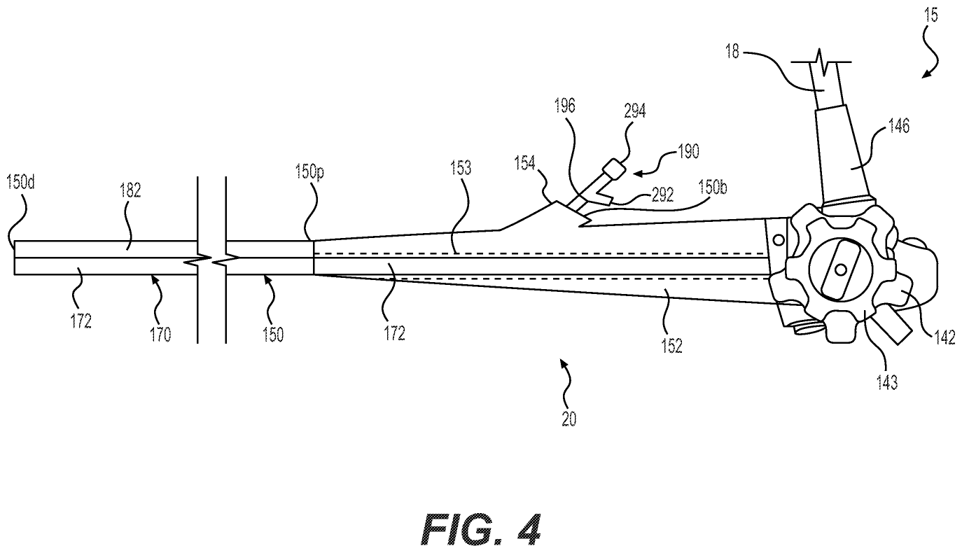

FIG. 4 shows another exemplary medical device system 15 including medical device 20. Medical device 20 is similar to medical device 10 in many respects, and like reference numerals refer to like parts. Like medical device 10, medical device 20 includes a handle 152 and a shaft 150. Handle 152 is similar to handle 52 and may include first and second actuators 142, 143, which control articulation of shaft 150. Handle 152 further includes a port 154 for introducing and/or removing tools, fluids, pressure (e.g., pulling negative pressure or suction) or other materials from the subject (e.g., patient) via a disposable portion 182 of shaft 150. Like handle 52, handle 152 may include one or more lumens (not shown) extending between port 154 and shaft 150, and said one or more lumens may define passages in communication with the various channels of shaft 150 (e.g., a working channel 185 and a second channel 187). Handle 152 also includes port 146 which may be detachably connected to an umbilicus 18 for introducing wiring for electronic components positioned at a distal end of shaft 150. Handle 152 includes a lumen 153 which may extend between an opening of port 146 and a proximal opening of a channel 183 of shaft 150 (discussed in further detail below). Thus, lumen 153 may define a passage of any suitable shape or dimension through which a removable portion 170 may be guided towards a channel 183 (discussed in further detail below). Thus, in some embodiments, umbilicus 18 may be detached from port 46 and removable portion 170 may be pulled proximally through an opening of port 146, thereby removing removable portion 170 and disposing the remainder of device 20, e.g., handle 152 and disposable portion 182.

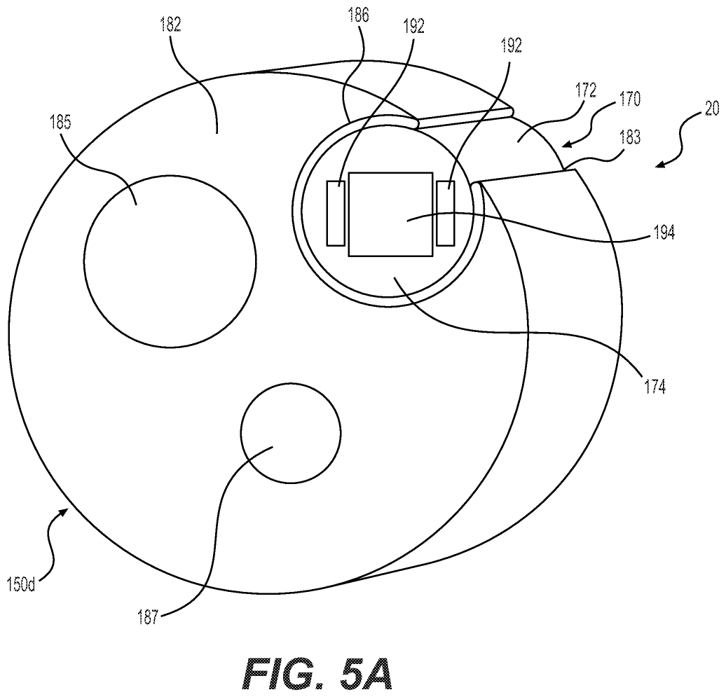

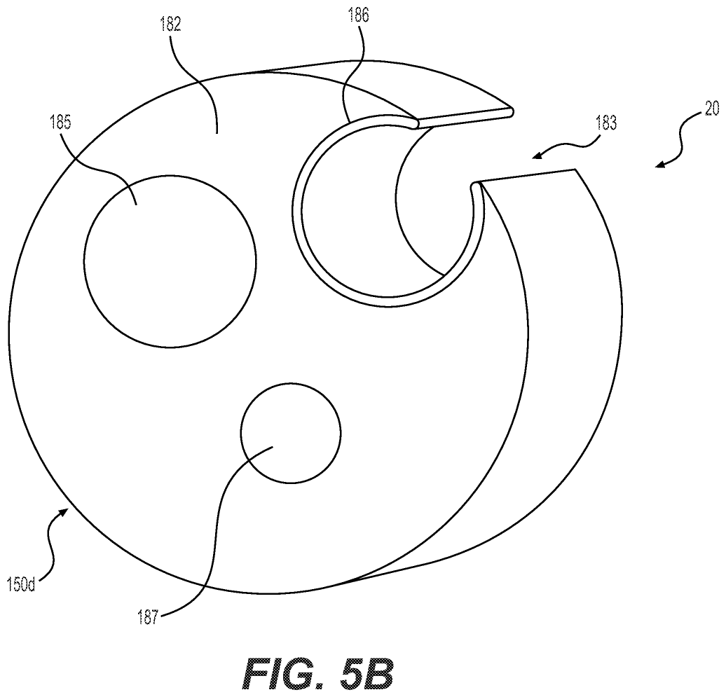

Shaft 150 includes a disposable portion 182 and a removable portion 170. Disposable portion 182 may extend between a distal end 150d and a proximal end 150p of shaft 150. Proximal end 150p may be coupled to a distal portion of handle 152 via any suitable means. Proximal end 150p may include one or more openings (not shown) which may be in communication with one or more lumens (not shown) extending from shaft 150 to various aspects of handle 152 (e.g., port 154, suction/fluidics valves (not shown), etc.) Disposable portion 182 is not particularly limited and may comprise any suitable biocompatible material. As shown in FIGS. 5A and 5B, unlike shaft 50 of device 10, disposable portion 182 includes a working channel 185 and a second channel 187 which may be any suitable channel configured to receive and/or deliver suction, fluid, etc. Disposable portion 182 is not necessarily limited to channels 185 and 187, and, in some examples, may include additional lumens or just one lumen, as well as additional features (e.g., an elevator feature). An arrangement of channels 185, 187 in FIGS. 5A and 5B is merely exemplary, and any suitable arrangement may be utilized. In some examples, disposable portion 182 may be devoid of any electronics or circuitry. Disposable portion 182, as shown in FIG. 4, may be in communication with port 154, via said one or more lumens, and port 154 may engage an adapter 190. Adapter 190 is not particularly limited and, in some examples, may include a first port 292, which may be in fluid communication with second channel 187, a second port 294, which may be in communication with working channel 185, and a connective end 196 engaging port 154. Disposable portion 182, along with handle 152 and adapter 190, may be disposable after procedure.

Disposable portion 182 may further include a longitudinal channel 183 (shown in FIGS. 5A and 5B), which may extend between distal end 150d and the proximal end 150p of shaft 150. Channel 183 may be in communication with port 146, via lumen 153, as discussed above. Channel 183 is not particularly limited and may be of any suitable dimension configured to receive an elongated portion 172 of removable portion 170. Like channel 83, channel 183 may be “partially open”, as shown in FIGS. 5A and 5B and discussed above with respect to channel 83, while still being configured to retain removable portion 170 by spanning around a sufficient degree/amount of a perimeter of elongated portion 172 (preventing elongated portion 172 from passing through the radial opening in disposable portion 182). In other examples, channel 183 may be closed thereby encapsulating a whole perimeter of elongated portion 172. The cross-section of channel 183 is also not particularly limited and may correspond to the cross-section of elongated portion 172. As shown in FIG. 5A, channel 183 may have a round, circular cross-section, but is not necessarily limited thereto. The cross-sectional shape of channel 183 may be keyed to/complementary/mating to the cross-sectional shape of elongated portion 172. In other examples, channel 183 may have a C-shaped, U-shaped, or teardrop-shaped cross-section, which may be suitable for retaining or clamping elongated portion 172. Like reusable portion 82, distal face 184 of disposable portion 182 may further include an abutment 186 outlining a distal opening of channel 183 that is configured to inhibit further distal translation of removable portion 170 past distal face 184. For example, abutment 86 may include a projection that extends inward along a perimeter of channel 183, effectively causing a distal opening of channel 183 to have a smaller perimeter than (a) more proximal portions of channel 183 and (b) elongated portion 172.

Removable portion 170 includes an elongated portion 172 that may extend throughout a length of channel 183 and a length of lumen 153 of handle 152, and within umbilicus 18. A material of elongated portion 172 is not particularly limited and may comprise any suitable biocompatible material. As shown in FIG. 4, elongated portion 172 may include a distal face 174 including various electronic features including one or more imager(s) (e.g., a camera) 194, and/or light features 192 (e.g., LEDs). Relative positions and sizes of imager 194 and light features 192 shown in FIG. 5A are merely exemplary, and any suitable arrangement may be utilized. The various cables/wirings associated with each of imager 194 and light features 192 may extend throughout a length of elongated portion 172. In some examples, elongated portion 172, as shown in FIG. 5A, may be without any channels or lumen configured to receive medical tools/instruments, suction, or fluidics, so that elongated portion 172 may be easily cleaned, sterilized, and re-used after procedure. In some examples, an inflatable liner may also surround a surface of elongated portion 172 (like elongated portion 72 shown in FIGS. 2C and 2D) and/or a surface of channel 183, which may assist in securing elongated portion 172 within channel 183 and removing elongated portion 172 from channel 183.

Unlike elongated portion 72, elongated portion 172 may fit within and extend throughout channel 183 via an opening of port 146, and a distal end of removable portion 170 may be secured by abutment 186. A proximal portion of removable portion 170 may extend within umbilicus 18 and a proximal portion of removable portion 170 may be coupled to any exemplary unit, processor, etc., which may support the various electronic features present on distal face 174. Removable portion 170 may also be completely removed from disposable portion 182 and handle 152 by pulling elongated portion 172 out of channel 183, lumen 153, and umbilicus 18, through port 146. Thus, a user may remove removable portion 170 after use in a procedure, and clean and sterilize removable portion 170 for re-use. Disposable portion 182 and handle 152 may be disposed of after use in a procedure, and the sterilized removable portion 170 may be inserted into a new disposable portion 182 and handle 152 for a subsequent procedure.

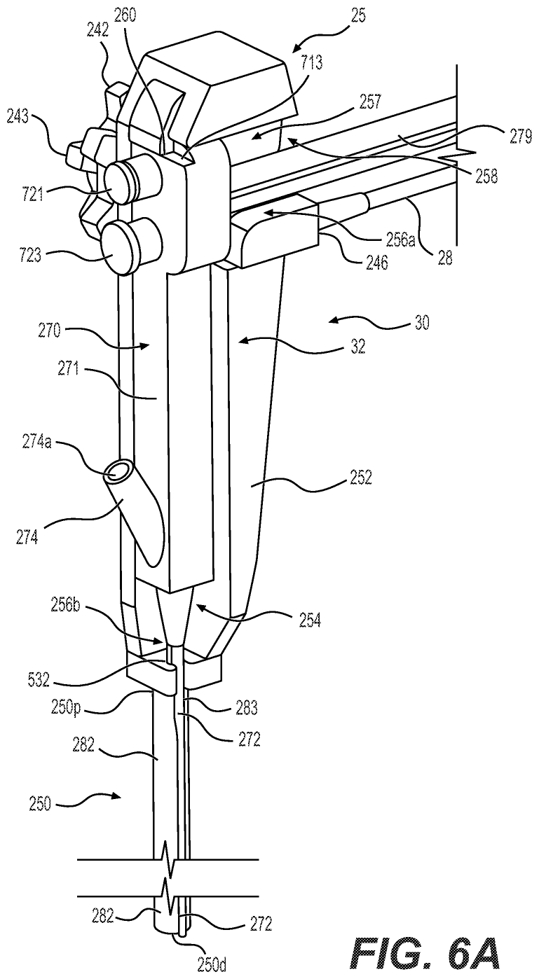

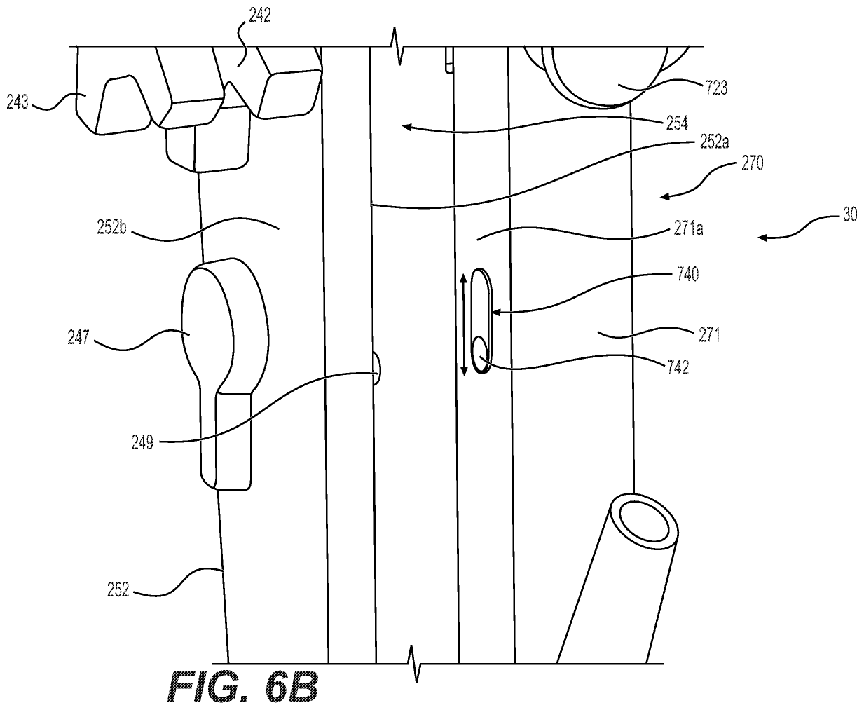

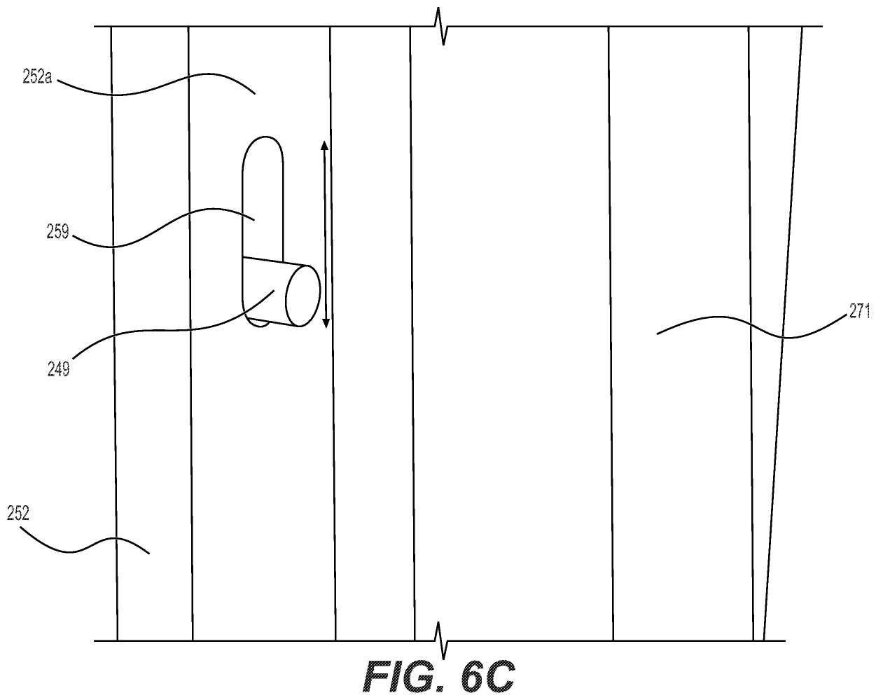

FIG. 6A shows another exemplary medical device system 25 including medical device 30. Medical device 30 is similar to medical device 10 in some respects, and like reference numerals refer to like parts, where feasible. Medical device 30 may include a reusable portion 32 and a disposable portion 270. Like medical device 10, reusable portion 32 of medical device 30 includes a handle 252 and a shaft 250. Handle 252 is similar to handle 52 and may include first and second actuators 242, 243, which control articulation of shaft 250. Handle 252 also includes port 246, which may be fixedly or detachably connected to an umbilicus 28 for introducing wiring for electronic components positioned at a distal end of shaft 250. Like handle 52, handle 252 may include one or more lumens (not shown) extending between port 246 and shaft 250, and said one or more lumens may define passages in communication with various channels of shaft 250 (not shown). Said one or more lumens may accommodate for various cables, wirings, circuitry, etc. extending within umbilicus 28 and through port 246.

Handle 252 includes a recess or cavity 254 that extends between a proximal portion 256a and a distal portion 256b of handle 252. Cavity 254 is not particularly limited and may be of any suitable dimension and/or shape that is configured to receive at least a portion of disposable portion 270. For example, as shown in FIG. 6A, cavity 254 may receive a body 271 of disposable portion 270. Moreover, cavity 254 may be partially open or exposed so that aspects of disposable portion 270, e.g., body 271, when within cavity 254, may be visible and accessible to a user. As shown in FIG. 6A, cavity 254 may have a longitudinal axis that extends approximately parallel or coaxial with a central longitudinal axis of handle 252/shaft 250. Cavity 254 may have an approximately square or approximately rectangular cross-section taken perpendicularly to the longitudinal axis of cavity 254, with two sides of the cavity being open. Walls of cavity 254 may have an approximately “L” shape at portions of cavity 254. A proximal portion of handle 252 may include a proximal opening or passage 257 extending from one side of handle 252 to the opposite side. Proximal passage 257 may have a longitudinal axis extending in a transverse direction relative to a central longitudinal axis of handle 252 and shaft 250. Proximal passage 257 may be configured to provide passage for a cable 279 extending from a proximal portion of disposable portion 270. An opening 258 of passage 257 through which cable 279 extends may be adjacent to port 246, such that umbilicus 28 and cable 279 extend away from handle 252 approximately perpendicularly. As shown in FIG. 6A, proximal passage 257 may be in fluid connection with cavity 254, thereby forming a continuous passage or space. Like cavity 254, passage 257 may also be partially open or exposed. Thus, passage 257 may expose cable 279 so that it may be visible and accessible to a user. For example, as shown in FIG. 6A, passage 257 may have an approximately square or approximately rectangular cross-section taken perpendicularly to the longitudinal axis of passage 257, with one side of the cavity being open. Walls of passage 257 may have a squared “U” shape.

The proximal portion of handle 252 may further include an edge 260, which is configured to protrude inwards towards/into cavity 254, but not necessarily limited thereto. Edge 260 is not particularly limited, and may be any suitable edge configured to engage a corresponding surface or ridge 713 of body 271, which is also not particularly limited, so that edge 260 and ridge 713 may form a snap-fit connection between handle 252 and disposable portion 270. However, the connection between handle 252 and disposable portion 270 is not limited to a snap-fit connection, and handle 252 and disposable portion 270 may respectively include features for any suitable coupling means (e.g., friction fit, hooks, latches, etc.).

A distal portion of handle 252 may include an opening or passage 532, which leads to channel 283 of shaft 250. Opening 532 is not particularly limited and may be any suitable shape and/or size configured to receive an elongated portion 272 of disposable portion 270. For example, opening 532 may be of an equal diameter, or about an equal diameter, as that of channel 283, which receives elongated portion 272, as discussed below.

Shaft 250 is similar to shaft 50, and may be identical in some instances such that shaft 250 may include all the features noted above with respect to shaft 50. For example, like shaft 50, shaft 250 includes a reusable portion 282 extending between a distal end 250d and a proximal end 250p, and a disposable portion 270. Proximal end 250p may be coupled to a distal portion of handle 52 via any suitable means, and is in connection with opening 532 of handle 252. Reusable portion 282 may include a distal face (not shown) including various electronic features including an imager (e.g., a camera), and/or light features (e.g., LED). The various cables/wirings associated with each of the aforementioned electronics may extend throughout a length of reusable portion 282, through the opening of proximal end 250p, through one or more lumens (not shown) or cavity 254 of handle 252, and towards port 246 and umbilicus 28. In some examples, reusable portion 282 may be without any channels or lumens configured to receive medical tools/instruments, suction, or fluidics, so that reusable portion 282 (along with handle 252) may be easily cleaned, sterilized, and re-used. Reusable portion 282 includes longitudinal channel 283 which may be of any suitable dimension configured to receive an elongated portion 272 of disposable portion 270. Like channel 83, channel 283 may be open on a radially outer side of channel 283, as well as at a distal end of channel 283. In some examples, a side of channel 283 may be open along an entire length of reusable portion 282, between proximal end 250p and distal end 250d. As a result, the partially open channel may provide a user an option of inserting elongated portion 272 of disposable portion 270 within cavity 254 handle 252 and pushing disposable portion 270 through channel 283 until disposable portion 270 reaches distal end 250d, or an option of inserting elongated portion 272 of disposable portion 270 at distal end 250d and pressing elongated portion 272 into channel 283 and body 271 within cavity 254. Alternatively, elongated portion 272 may be inserted through an open side of channel 283, rather than advancing elongated portion 272 proximally or distally. Channel 283 may be rigid enough to hold disposable portion 270 in place, but flexible enough to allow disposable portion 270 to be pressed into channel 283 with enough force. Also, the partially open channel 283 may provide design flexibility in disposable portion 270 such that the size and shape of elongated portion 272 is not constrained by a closed channel. However, in other examples, channel 283 may be closed thereby encapsulating a whole perimeter (e.g., a whole circumference where elongated portion 272 has a circular cross-section) of elongated portion 272.

Like disposable portion 70, disposable portion 270 may be devoid of any electronics or circuitry, and disposable portion 270 may be disposable after procedure. Disposable portion 270 includes body 271 received by cavity 254, and elongated portion 272 which may extend throughout a length of channel 283.

Body 271 is of any suitable shape or dimension configured to be received within cavity 254 of handle 252. Body 271 includes a port 274. Port 274 is not particularly limited, and may be any suitable port, e.g., a Y-port, including an opening 274a for introducing and/or removing tools or other materials from the subject (e.g., patient). Body 271 may include a lumen (not shown) which may extend between opening 274a of port 274 and a proximal opening of a channel of elongated portion 272. A proximal portion of body 271 may further include at least one cable 279 and additional controls or buttons 721 and 723. Buttons 721 and 723 may be used to control, for example, fluidics (e.g., air, water, or suction) and/or electronics (e.g., light brightness, image capture, or other features). Cable 279 is not particularly limited and may include one or more channels configured to receive fluids and/or pressure (e.g., pulling negative pressure or suction) from a fluid and/or pressure source and/or wires/cables. Thus, cable 279 may extend between a proximal portion of body 271 (so it may pass through proximal passage 257 of handle 252) and a fluid and/or pressure source. Said one or more channels of cable 279 may be in fluid communication with one or more respective lumens of body 271 (not shown), which may extend between distal opening(s) (not shown) of cable 279 and proximal opening(s) of corresponding channels extending throughout a length of elongated portion 272. Controls 721, 732 are not particularly limited and may be any suitable actuators or valves configured to control delivery of suction or fluid to a distal end of elongated portion 272.

Elongated portion 272 is not particularly limited, and may be similar or identical to elongated portion 72, 72′. Like elongated portion 72, elongated portion 272 may include an elevator feature (not shown) for changing an orientation of a medical instrument inserted within a channel of elongated portion 272. Said elevator may be pivotable via an actuation wire or another control element (not shown) that extends throughout a length of elongated portion 272 (e.g., via a separate lumen of elongated portion 272) and to a respective actuator positioned on handle 252 or body 271 of removable portion 270.

FIGS. 6B and 6C illustrate an exemplary actuation mechanism of said elevator feature. As shown in FIG. 6B, removable portion 270 may further include a slider 740 on body 271. Slider 740 includes a through-hole 742 which is configured to receive a corresponding actuating peg, e.g., a peg 249. Slider 740 may extend/be movable longitudinally in the same direction as a longitudinal axis of body 271, and may be positioned on a surface 271a of body 271 that is adjacent to a surface 252a of handle 252, which may be an interior surface of the side of handle 252 (e.g., a wall of cavity 254). An exterior surface 252b of the side of handle 252 may include a lever 247 (discussed in further detail below). Surface 252a and surface 252b may be interior and exterior surfaces, respectively, of a same side of handle 252. Slider 740 may be an actuatable feature that is coupled to, or in engagement with, an actuation wire or another control element associated with the elevator feature of elongated portion 720. Slider 740 is not particularly limited, and may be any suitable feature configured to translate in a first direction, e.g., a proximal direction, and a second direction, e.g., a distal direction, as represented by the directional arrow shown in FIG. 6B. Moreover, slider 740 may be biased towards one direction, via any suitable mechanism (e.g., a spring bias). For example, as slider 740 translates in a first direction, said elevator feature may orient a medical instrument within elongated portion 272 in a first orientation, and when slider 740 translates in a second direction (opposite the first direction), said elevator feature may orient the medical instrument in a second orientation. In some aspects, moving slider 740 in the first direction may raise the elevator, and moving slider 740 in the second direction may lower the elevator.

Handle 252 includes an actuator configured to engage slider 740. The actuator is not particularly limited, and may be, for example, lever 247. Lever 247 may be positioned on a side of handle 252 also including actuators 242, 243 for ergonomic reasons, but is not necessarily limited thereto. Actuators 242, 243 may be knobs. Lever 247 is coupled to peg 249, which may extend through a wall of handle 252 to interior surface 252a, as shown in FIGS. 6B and 6C. Moreover, peg 249 may reside within a longitudinal slot 259 extending in the same direction as slider 740. The length of slot 259 is not particularly limited, and may correspond to a distance slider 740 may translate in the first and second direction. Peg 249 may also translate within slot 259 via an actuation of lever 247, as represented by the directional arrow shown in FIG. 6C. The translational direction of peg 249 may correspond to the translational direction of slider 740 so that, when peg 249 is engaged within through-hole 742, the translation of peg 249 may simultaneously translate slider 740, thereby actuating the elevator feature.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical device, comprising:
a handle including a port;
a shaft extending distally from the handle, wherein the shaft includes a channel extending along at least a portion of a length of the shaft, wherein the channel includes a longitudinal opening on a radially outer surface of the shaft; and
a removable portion extending through the channel, wherein the removable portion includes a first lumen, and wherein the removable portion is configured to be separated from the handle and the shaft.

2. The medical device of claim 1, wherein the channel of the shaft is off center from a central longitudinal axis of the shaft.

3. The medical device of claim 2, wherein the channel is configured to prevent the removable portion from passing through the longitudinal opening of the channel.

4. The medical device of claim 1, wherein the longitudinal opening of channel includes a notch, a groove, or a slit extending along an entire length of the shaft.

5. The medical device of claim 1, wherein the shaft includes a distal end, wherein the distal end includes an abutment outlining a distal opening of the channel, and wherein the abutment is configured to inhibit distal translation of the removable portion past the distal end.

6. The medical device of claim 1, wherein the removable portion includes a second lumen.

7. The medical device of claim 1, wherein the removable portion consists of the first lumen and a second lumen.

8. The medical device of claim 1, wherein the removable portion includes a proximal adapter, wherein the proximal adapter includes a first port in fluid communication with the first lumen of the removable portion.

9. The medical device of claim 8, wherein the proximal adapter is configured to releasably engage the port of the handle.

10. The medical device of claim 9, wherein the proximal adapter includes an actuator configured to release the proximal adapter from the port of the handle.

11. The medical device of claim 9, wherein the proximal adapter is a y-shaped adapter including the first port of the proximal adapter and a second port of the proximal adapter.

12. The medical device of claim 1, wherein the first lumen includes an elevator configured to adjust an orientation of a tool or instrument inserted within the first lumen.

13. The medical device of claim 1, wherein the removable portion comprises a substantially teardrop-shaped cross-section.

14. The medical device of claim 1, wherein the removable portion includes a second lumen, and wherein only the removable portion is configured to receive fluid via the first lumen and/or the second lumen.

15. The medical device of claim 1, wherein the removable portion is configured to be completely removed from the handle and the shaft by pulling the removable portion proximally from the port of the handle.

16. A medical device, comprising:
a handle including a cavity;
a shaft extending distally from the handle, wherein the shaft includes a channel extending along at least a portion of a length of the shaft, wherein the channel includes a longitudinal opening on a radially outer surface of the shaft; and
a removable portion extending through the cavity and the channel, wherein the removable portion includes a first lumen, and wherein the removable portion is configured to be separated from the handle and the shaft.

17. The medical device of claim 16, wherein the cavity is partially open thereby exposing at least a portion of the removable portion.

18. The medical device of claim 17, wherein the removable portion includes a body and an elongated portion, and wherein the body includes a port configured to receive a medical instrument.

19. A medical device, comprising:
a handle including a port;
a shaft extending distally from the handle, wherein the shaft includes a channel extending along at least a portion of a length of the shaft; and
a removable portion extending throughout the channel, wherein the removable portion includes at least one lumen, and wherein the removable portion is configured to be removed from the handle and the shaft via the port; and
a liner positioned between the channel and the removable portion, wherein the liner is configured to transition between a deflated state and an inflated state.

20. The medical device of claim 19, wherein the liner is fixed to at least a portion of a surface of the removable portion and wherein the removable portion and the liner are configured to be completely removed from the handle and the shaft by pulling the removable portion proximally from the port of the handle.
